(19) United States
(12) Patent Application Publication
(10) Pub. No.: US 2004/0236076 A1
(43) Pub. Date: Nov. 25, 2004

(54) Immunoconjugates made of egg-yolk antibodies (igy), production and use thereof in diagnoses and therapy

(76) Inventors: Wolfgang Bergter, Hanover (DE); Hartmut Kobilke, Damsdorf (DE); Joachim Sroka, Damsdorf (DE)

Correspondence Address: Law Offices of Karl Hormann, P O Box 381'516, Cambridge, MA, 02238-1516, US

(21) Appl. No.: 10/478,381

(22) PCT Filed: May 3, 2002
(86) PCT No.: PCT/DE02/01606

(30) Foreign Application Priority Data
May 15, 2001 (DE) 10123505.4

(51) Int. Cl. C07K 16/18

(52) U.S. Cl. 530/387.1

(57) ABSTRACT

The present invention relates to immunoconjugates from egg-yolk antibodies (IgY), their production and application in diagnostics and therapy. It is an object of the present invention to propose alternative products to conventional immunoconjugates for diagnostic and therapeutic use. The immunoconjugates in accordance with the invention are superior to monoclonal antibodies and immunosera from mammals, as well as to the IgY compounds from conventional chickens, in respect of purity and antigen targeting. The production of human anti-IgY-antibodies is to be avoided if possible, and the immunoconjugates are to be producible economically in a manner doing justice to animal protection and in large quantities. In accordance with the invention, the object is accomplished by polyclonal IgY conjugates from intact egg-yolk antibodies (IgY), IgY fragments, Fab constructs or humanized egg-yolk antibodies from SPF-chickens, preferably transgenic SPF-chickens. The antigens or antigen fragments are to be conjugated as immunologically effective component with at least one further component which may be a signal agent, an active agent or a booster molecule.

The present invention relates to IgY conjugates made from egg-yolk antibodies (IgY) and their production as diagnostic and therapeutic agents in accordance with the characteristics of claims 1 and 13.

For a better understanding of the invention, the terms of art as they appear in the application will be defined in greater detail and in the context of this application they shall have no other meaning.

“Specified Pathogen-Free Chickens” (in short “SPF-Chickens”):

These are understood to be animals descended in accordance with the Guidelines of the European Pharmakopoeia and DAB10 from SPF-mothers and which from their first day of life have been maintained under SPF conditions. These animals are free of human and fowl-pathogen causative agents and their antibodies.

“IgY” (Immunoglobulin Yolk):

These define immunoglobulins which are extracted from the egg-yolk of fowl eggs and which correspond to the IgG in the serum of the chickens. These aviary immunoglobulins are structurally distinguished from the mammalian IgG primarily by their higher molecular weight resulting from a greater number of constant regions in the Fc fragment.

“Specific IgY”:

In the present context it is defined as the proportion of the entire IgY which recognizes the antigen used for immunization, whilst unspecific IgY defines that proportion of the entire IgY which is formed independently of the immunization by contact of the animals with antigenically acting materials or non-pathogenic and pathogenic causative agents.

“Intact Antibodies”:

These shall be understood to be immunoglobulins which are not fragmented, i.e., they possess an Fc fragment and two Fab segments, Fc including the constant regions of the heavy chains and Fab including the variable regions of the heavy and light chains.

“IgY Fragments”:

In the present context, these connote Fab fragments of aviary immunoglobulins which have no or a small residue of the Fc fragments. For the sake of simplicity, the term “Fab” will hereafter be understood also to mean such fragments as F(ab)₂.

“Fab Construct”:

The term “Fab construct” is to define synthetic formations of two or more different Fab fragments (bi- or tri-valent constructs, etc.) which recognize one or more different antigen determinant sites (mono-, bi- or tri-specific constructs, etc.) and which may thus satisfy one or several functions, i.e. they act mono-, bi- or tri-functionally.

“Antigen Determinant Site”:

This is that molecular structure on an antigen which is specifically recognized by an antibody (also called “epitope”).

“IgY Compound”:

These are to be understood to be the egg-yolk antibodies (IgY), IgY fragments, Fab constructs or chimeric egg-yolk antibodies extracted from egg-yolk and, if required, purified.

“Signal Agents”:

These are understood to be materials which allow an analytical, visual or image-forming evaluation of pathologic or physiologic results.

“Active agent”:

This describes agents which may be used therapeutically, e.g. enzymes, antibiotics, virostatics and cytotoxins such as toxins or radionuclides. This does not exclude active agents which also fulfill the quality of a signal agent.

“Prodrug”:

This defines materials which are transformed in situ from an inactive pre-stage within the body to an active agent.

“Booster” and “Replenishing Immunization”:

This term will be mentioned in connection with the immunization of SPF-chickens. The boosters serve to attain the maximum IgY-titer. Replenishing immunization connotes the renewed antigen exposure by which the maximum IgY-titer is maintained until the end of the laying period.

Immunoconjugates based on monoclonal antibodies from mice (murine MAK) or from humans (human MAK) are known. Many methods have been established of conjugating signal agents (stains, fluorochromes, diagnostic radionuclides) or active agents (e.g. toxins, therapeutic radionuclides, photosensitizers, prodrugs) to these antibodies. They make possible examinations such as ELISA, RIA, fluorescent microscopy, impulse cytometry, scintigraphies, SPECT (single photon emission computed tomography), PET (positron emission tomography) and photo immunodiagnostics, or treatments such as radiation immunotherapy, immuno toxin therapy or photo dynamic therapy.

Among the disadvantages of the monoclonal antibody technology are mainly the monospecificity of the antibodies as well as the relatively complex and expensive development and confectioning of the monoclonal antibodies. In general, their confectioning must be carried out in fermenters since production in the ascites of mice has been largely proscribed in many countries.

Polyclonal immunosera of mammals (sheep, horse, pig, etc.) and immunized/infected donors (e.g. from the blood of HIV infected persons of high p24-antibody titers) are important in various diagnostic processes and passive immunotherapies. From the point of view of improved targeting of complex antigens the monoclonality of these immunosera is useful. However, immunosera of mammals or conventional chickens contain a relatively poorly defined polyspecific mixture of antibodies wherein the desired specific antibodies constitute the minor portion whereas the unspecific and, hence, undesired antibodies constitute the major portion. From the point of view of protecting animals, the production of immunosera from mammals is hardly acceptable, and for that reason it has been largely prohibited in several countries. The production of immunosera from human blood donations is expensive, by no means free of risks (transmission of viruses such as HIV, HCV and, possibly, prions), and is ethically problematic, and, for that reason, it is not practicable for a broad range of applications.

Polyclonal antibodies of aviary origin, among these egg-yolk antibodies (IgY) are also known. Currently, IgY is being offered by different manufacturers for in vitro diagnostics as an alternative to immunosera from mammals or monoclonal antibodies. Specialized literature also refers to examples of how egg-yolk antibodies can be coupled to biotin, FITC or horse radish peroxidase (POD) in order to render them useful for in vitro diagnostics (Schade et al., 2001 [1]).

The oral application of aviary antibodies from conventional chickens is also known. The parenteral application in human medicine has hitherto be been largely avoided, on the one hand because in mammals IgY, because of the phylogenic spacing, does not couple either to complement factor C1 or Fc receptors so that it cannot interact effectively with the defensive system of mammals, and, on the other hand, because IgY may cause allergic reactions. Furthermore, the production of IgY from egg-yolks from conventional chickens is not efficient and in respect of purity and affinity the resultant products are of inferior quality. The reason for this is that conventional chickens form numerous unspecific antibodies against other germs, and the response to the target immunogen is thus relatively weak and of short duration. For that reason, in the immunization of conventional chickens the proportion of specific IgY relative to the entire IgY is significantly less than 10% (U.S. Pat. No. 4,550,019; Hansen et al., 1998 [2]; Bouhours et al., 1998 [3]; Gassmann et al., 1990 [4]).

The closest state of the art is described in German patent specification 195 04 755 which relates to a passive immunotherapy of HIV infection by IgY. The IgY is targeted at the HIV-core-antigens p24 and p17 of specified pathogen-free (SPF-) chickens. Passive immunotherapies with human immunosera are known as well. Compared to immunosera and monoclonal antibodies IgY may be produced easily and inexpensively. SPF-chickens under the immunization protocol described in Example 2, after basic immunization and boostering will lay 5 to 7 eggs every week, up to their 16^{th} month of life. In this connection it was found that about 15,000 mg of specific IgY could be produced from the 250 to 350 eggs of a SPF-chicken, since, empirically, one egg yolk contains at least 50 mg of IgY of the desired specificity. Moreover, the pollution of SPF-chickens, because of the specified treatment prescribed for these animals, is significantly lower than in common chickens raised for meat and eggs. Consequently, the usual active immunizations, including two prescribed inoculations, against up to nineteen commercially significant infectious diseases common in the raising of conventional chickens are avoided. These immunizations account for the low quality of the IgY compounds of conventional chickens and for the complexity and expense of isolating specific IgY. The keeping of SPF-chickens is free of human and fowl pathogenic germs. For excluding such germs feed, water and immunization status are controlled regularly. No antibiotics are added to the feed in order to prevent concealment of the animals' SPF status.

From a medical point of view, the advantages of IgY in general reside in the recognition of several antigenic determinants by the specific antibodies, which ensures improved targeting of the target antigen, and in the fact that the antibodies are from fowl the immune system of which differs from that of mammals and humans as regards the antigen recognition. This also enhances the targeting of the target antigen since the aviary immune system recognizes (phylogenetically conditioned) antigenic determinants different from the human immune system. Accordingly, there is reduced competition for epitopes between aviary antibodies in vivo and the naturally present antibodies of a patient. Moreover, the assumed disadvantage of aviary antigens reacting with neither the human complement system nor with Fc receptors, protein A or protein G, may in many cases be shown to be insignificant or even as advantageous.

It is thus an object of the invention to develop and make available alternative products to conventional immunoconjugates which are suitable for diagnostic applications and therapeutic uses and which are superior in respect of their purity and antigen-targeting to monoclonal antibodies and immunosera from mammals as well as to IgY compounds from conventional chickens. In addition, the formation of human anti-IgY-antibodies is to be substantially avoided, and the immunoconjugates are to be made cost-efficiently in large quantities in conformity with animal protection policies.

Before describing the description of the invention, further terms will hereafter be defined as they are understood in the ensuing description of the invention as well as in the patent claims. In the context of this application they shall have no other meaning.

“Radio Immunoconjugate”

In this application this connotes compounds of IgY preparations of SPF-chickens or transgenic SPF-chickens which are conjugated with diagnostic and/or therapeutic radionuclides and/or booster molecules and which are used in the context of nuclear medical diagnostics and/or radiation immunotherapy.

“Immuno Toxin”

These are preparations of IgY compounds conjugated with toxins (vegetable toxin, cytostatics, chemotherapeutics, antibiotics, etc.) and which are used in the context of immunotoxin therapy.

“Specific Targeting”

This defines a process in which the signal or active agent is conjugated to the desired target antigen by means of the immunoglobulin.

“Pretargeting”

In this context, it embraces applications which cause a boost of signals or effects of the IgY compounds by interposing further antibodies of another species or other suitable molecules (compiled herein also under the generic term “booster molecule” in any phase between antigen connection and signal or active agent.

“Chimeric Egg-Yolk Antibodies”

These mean

i) humanized immunoglobulins in which Fab fragments of a chicken are biochemically connected to Fc fragments of human IgG, or

ii) humanized immunoglobulins from transgenic SPF-chickens in which the gene sections of the constant regions of IgY are more or less completely replaced by the constant regions of human IgG.

“IgY conjugate”

In this context, this term embraces compounds of polyclonal IgY preparations (immunological component) and signal agents and/or active agents (diagnostic or therapeutic component) and/or booster molecules.

In accordance with the invention the object is accomplished by polyclonal IgY conjugates from intact egg-yolks antibodies (IgY), IgY fragments, Fab constructs or humanized egg-yolk antibodies from SPF-chickens, preferably from transgenic SPF-chickens,. As an immunologically effective component, the antibodies or antibody fragments are to be conjugates with at least one other component which may be a signal agent, an active agent of a booster molecule.

The invention relates to IgY conjugates from egg-yolk antibodies (IgY) and their production as diagnostic and therapeutic agents and well as for experimental applications in animals (e.g. mouse, sheep or non-human primates). Its use is not limited to the parenteral application, but, depending upon the application target, it includes external (e.g. in a melanoma) and enteral applications (e.g. cancer of the oesophagus).

Moreover, the invention relates to the confectioning of the IgY conjugates as ready-to-use diagnostics or medicines, either as individual components, as labeling kits or as ready-to-use compounds. Target antigens under consideration are all the proteins, peptides, glycoproteins, nucleic acid, polysaccharides and lipoproteins. Above all, the invention relates to IgY conjugates against tumor antigens, hormones, receptor proteins, RNS- and DNS sections, infectious germs, prions or part of the antigenic determinants of these target antigens. Depending upon the complexity of the antigen used for the immunization, the IgY conjugates may contain more or less polyspecific IgY.

A further aspect of the invention relates to pretargeting methods in which either

i) biotinylated IgY connects to the target antigen and serves as an intermediary for labeled avidin or streptavidin, or

ii) unconjugated IgY connects to the target antigen and serves as an intermediary for human or humanized anti-IgY conjugates.

In the case of i) the strong bond between biotin and avidin or streptavidin is used so the biotin component in the IgY conjugate may also be called booster molecule.

Finally, the present invention relates to compounds and applications of the IgY conjugates in accordance with the invention by which their diagnostic and/or therapeutic properties may be improved, such as surgical procedures, chemotherapeutics, immunomodulators and paraimmunity inducers.

The components related directly to the invention, their functions, effects and the application of the IgY conjugates in accordance with the invention will hereafter be set forth in greater detail and the solutions will be presented in detail.

a) Immunological Component

In accordance with the invention, the immunologically effective components consists either of

i) intact egg-yolk antibodies (IgY), IgY fragments, Fab constructs or

ii) chimeric egg-yolk antibodies, i.e. biochemically humanized IgY or, preferably, genetechnologically humanized IgY from eggs of transgenic SPF-chickens.

In this context, the Fab fragments may be mono-, bi- or tri-valent and may be effective in a mono-, bi- or tri-specific manner (i.e. they may recognize one, two or different antigenic determinants). The Fab fragments may be produced by enzymatic digestion. Chimeric antibodies may be produced by splicing the aviary fragment to the Fc fragment of another organism (in this context preferably human Fc fragment). Humanized IgY in which only the hyper-variable regions come from chicken IgY and all constant regions are derived from human IgG, can be produced in transgenic chickens. With chimeric antibodies it is not only possible to reduce or prevent the formation of undesired antibodies against IgY, but it also makes possible the use of the effector mechanisms of the constant regions of human IgG.

b) Signal Agents

The diagnostic component contained in the inventive IgY conjugates are signal agents such as radionuclides (gamma emitters such as technetium 99m, indium 111, iodine 123, iodine 125, iodine 131, thallium 201, selenium 75, gallium 67, xenon 133), enzymes (such as peroxidase, alkaline phosphatase, and galactosidase), photosensitizers (porphyrin derivatives etc.) or stains (e.g. fluorochrome, colloidal gold). Depending upon the purpose manner of application, affinity of the antibodies and radionuclides, activities of 10-1850 MBq (0.27-50 mCi) are necessary for scintigraphic diagnostics. In particular, for iodine 131, for instance, 37-370 MBq (1-10 mCi) (preferably 37-74 Mbq) may be considered; for iodine 123, 185-370 MBq (5-10 mCi); for technetium 99m, 74-1480 MBq (2-40 mCi) (preferably 74-740 MBq (2-20 mCi)); for indium 111 and gallium 67, 74-185 MBq (2-5 mCi), or for selenium 75 about 10 MBq (0.27 mCi).

c) Active agents

As a therapeutic component, the inventive IgY conjugates may alternatively or additionally contain radionuclides (beta emitters, alpha emitters or Auger electron emitters), e.g. phosphorus 32, strontium 89, yttrium 90, iodine 125, iodine 131, samarium 153, erbium 169, rhenium 186, rhenium 188, krypton 85, holmium 166, astatine 21, bismuth 212, bismuth 213, radium 224, actinium 225. As in diagnostics, the required activities in radiation, immunotheraphy are dependent upon several factors: the kind of illness, radiation sensitivity of the diseased cells, mass of the cells to be treated, extent and spread pattern, antigen expression, the manner of application, affinity of the antibodies and the energy spectrum of the radionuclide. For iodine 131, for instance, activities may be considered in a broad range of from 370 MBq to 11.1 GBq (10-300 mCi). In general, these activities are applied once, but may take place fractionally at intervals of several weeks or months (preferably 3-6 weeks) or, upon the occurrence of a relapse, it may be repeated up to a total dose of 40-80 GBq (1-2 Ci). The conjugation of tumor-specific IgY with cytostatics may reduce the disadvantages of conventional chemotherapies. One of the greatest problems in oncology is the limited diagnostic certainty in staging examinations (in particular the lack of techniques for proving or excluding the presence of micrometastases) as well as the low therapeutic spectrum of the cytostatics as a result of their high toxicity and simultaneous low specificity. The factors over and over again lead to a significant uncertainty relative to the therapeutic procedure. A specific cytostatis of lower side effects as a result of antibodies would facilitate the decision regarding chemotherapy and would, therefore, contribute to reducing complex, expensive and/or stressful staging examinations. In the sense of the invention the egg-yolk antibodies may also be conjugated with different cytostatics (e.g. alkylants, vincaalkaloids, intercalary antibiotics, antimetabolites, Taxol®). The inventive IgY conjugates may also contain toxins as therapeutically effective components (ricin A, abrin, pseudomonas exotoxin, diphtheria toxin, gelonin and others). Chemotherapeutic agents (cytostatics, antibiotics, antiviral substances), fibrinolytes (e.g. streptokinase, urokinase, rt-PA), enzymes for activating prodrugs (e.g. bacterial carboxy peptidase CPG2 which catalyzes the hydrolytic split of reduced and non-reduced folate) or prodrugs themselves are also suitable therapeutic components in the inventive IgY conjugates. Finally, the inventive IgY conjugates may contain photosensitive substances as effective components for selectively destroying diseased cells by way of a photodynamic therapy. Photosensitizers may be, for instance, Photofrin®, 5-aminolevulinic acid or Foscan®.

d) Booster Molecules

For diagnostic and therapeutic applications, the IgY conjugates produced in accordance with the invention may contain biotin in order on the basis of avidin-biotin or streptavidin-biotin-bonds to boost their signal or effect (so-called pre-targeting). In addition, signal and effect of the IgY conjugates may be boosted by further components which are conjugated to the IgY molecule (e.g. complement).

e) Adjuvants, Complementary Measures

For optimizing the target of the therapy, the IgY conjugates in accordance with the invention may be combined with other active agents. Aviary antibodies may initiate the formation of human anti-IgY antibodies. Repeated applications of IgY conjugates thus result in neutralizing the diagnostically and therapeutically applied antibodies or even in allergic reactions. This can be prevented by suppressing the immune system with Cyclosporin®, for instance. On the other hand, the use of radioactive iodine may lead to an undesired radioactive exposure of the thyroid gland. For that reason, it is advisable in such cases, prior to a radiation immunotherapy with IgY-iodine 131 to block the function of the thyroid gland by administering potassium iodide or sodium perchlorate. In an immunotherapy of viral infections, free virus particles would trap IgY conjugates which would thus no longer be available for the specific targeting of cells replicating the virus. It is, therefore, advisable to reduce the viral replication by a pre-treatment with one or more antivirally or antiretrovirally effective chemotherapeutics and, if necessary, to trigger or induce the renewed antigen expression by temporarily not administering this medication. In addition, the release of the viral particles could be reduced by means which stabilize the cell membrane (e.g. interferon-alpha). On the other hand, it may be necessary, in dormant viral infections (e.g. HIV or EBV infections) to stimulate the antigen expression of the infected cells (e.g. by use of lnterleukin-2®) or to increase the effectiveness of the IgY conjugates by other antiviral agents (e.g. Interferon-alpha® and/or Ribavirin® in the treatment of hepatitis C). In the treatment of bacterial infections it may also be advisable complementingly to apply further active agents (antibiotics). By analogy, in oncological illnesses it may be necessary to supplement a radiation immunotherapy by administering cytostatics or by an immunotoxin therapy by localized external radiation. In general, immunomodulators or paraimmunity inducers may favorably influence the effect of a radiation immunotherapy, immunotoxin therapy or photodynamic therapy.

f) Labeling Processes

Numerous established methods have for years been available for radioactively labeling murine and human monoclonal antibodies (Peters J. H., Baumgarten H. (Eds.): Monoclonal Antibodies, Springer-Verlag, Berlin 1992). Metallic radionuclides such as technetium 99 m, indium 111 or astatine 211 are usually connected to the antibodies by complexing agents -e.g. diethylene triamine pentaacetic acid (DPTA) or deferoxamine (DFO)- or by partial reduction of the disulfide bridges of the antibody (e.g. by 2-mercaptoethanol). Many radioactive iodine isotopes may also be conjugated to activated aromatic groups of the antibodies (e.g. the phenol ring of tyrosine) by direct electrophilic substitution. Potassium iodate (KIO₃), dissolved chloramine T or chloramine T tied (iodobeads) to carriers (e.g. polystyrene beads) or iodogen (1,2,3,4,6-tetrachloro-3α,6α-diphenyl-glyco uril) are usually used as oxidizing agents for forming iodine cations. Each individual antibody may be labeled with at least one radioactive atom. To achieve higher affinity without adversely affecting the specific bonding capability of the antibodies, the conjugation may take place in the presence of the target antigen which is subsequently separated again, so that the bond of the radionuclide in the range of the hypervariable regions is prevented. Chromium fluorides, photosensitizers, cytostatics, toxins may be conjugated to antibodies by processes based upon the reactions of the amino groups of the antibodies, in part also by commercially available spacers of by the insertion of SH groups into the antibodies. Other processes are based upon splitting the antibodies and release of SH groups to which the signal and active agents may be conjugated. In addition, the carbohydrate residue of the antibodies may be oxidized by periodate which leads to aldehyde groups by means of which the signal and active agents can be conjugated. Finally, bi- and tri-functional Fab constructs may by an active agent be bonded to one or two Fab fragments of the molecule and dock onto the target antigen by means of the free Fab fragment.

g) Confectioning

In the context of the invention, labeling kits (preparation kits) with IgY compounds may be offered for radio immunoconjugates so that they may be labeled in situ by the user with the usually short-lived radionuclide. Given a sufficiently long half life, the IgY compounds conjugated with a radionuclide (i.e. complete radio immunoconjugates) may also be directly furnished to the user. In the labeling kits or complete compounds, the IgY compounds or ready-made radio immunoconjugates are available as a sterile isotonic injection solution, if necessary in a suitable buffer (preferably PBS, pH 7.4) with an appropriate additive (e.g. stabilizers) and suitably packaged (plastic/glass ampules/piercing vial, and, if necessary lead containers, deep cooled at +4 to −20° C.). In labeling kits, the IgY compound will be in the first ampule/piercing vial and the reducing agent (e.g. when labeling with technetium 99m) or the oxidizing agent (e.g. when labeling with iodine 131) will be in a second ampule/piercing vial. For purifying the radio immunoconjugate, each labeling kit may contain a chromatographic column and, for quality control, a thin-layer chromatography set. Depending upon given requirements, the labeling kits may contain the IgY compounds or the ready-made IgY conjugates for one or several applications.

In accordance with the invention the following areas of use and applications are provided:

a) Areas of use in general: The IgY compounds in accordance with the invention can react with a plurality of antigens: with physiological molecules, with tumor-specific antigens, with infectious germs and their antigens, with prions as well as with RNS- or DNS sections or with the molecules associated therewith. Depending upon the signal or active agent (radionuclide, enzyme, febrinolytic, stain, photosensitizer, toxin) the inventive IgY conjugates can be used for in vitro and for in vivo diagnostics and/or therapy of tumors, infections, clotting defects and autoimmune diseases.

b) Physiological Target Antigens: Among the molecules which may be considered for the inventive IgY conjugates are leucocyte antigens in accordance with the CD classification (antigens of NK-cells, B-cell-antigens, myeloic antigens, progenitor antigens, activation antigens, adhesion antigens, cytokin receptors) as well as intra-cellular molecules (e.g. cyto keratins), receptors (androgen-, estrogen-, dopamin-D2- or somatostatin receptors, etc.) or, for instance, clotting factors (e.g. fibrin or plasminogen).

c) Pathological Target Antigens: Among the tumor-specific antigens which may be considered for IgY conjugates, are predetermined hyperexpressed leucocyte antigens (e.g. CD20, CD22), hyperexpressed androgen and estrogen receptors, calcitonin, thyreoglobulin, as well as, in a more narrow sense, tumor label such as Egp 34, Ca 15-3, sialyl-Le^{a}-antigen, BCA 225, melanoma associated antigens (MAA), CEA, 17-1A, PAP, neuron-specific enolase (NSE), terminal desoxynucleotidyl-transferase (TdT), bromodesoxyuridine (BrdU), Ki 67, PCNA, myeloperoxidase (MPO) and mutated p53. Amon the infectious germs which may be targeted by the IgY conjugates of the present invention, are bacteria (bacillus anthracis, borrelia, brucella, mycobacteria, pseudomonas aeroginosa, salmonella, staphylococci, toxoplasma gondii, treponema, trypanosoma), viruses (HIV-1, HIV-2, HTLV-1, HTLV-2, HCV and other flavi viruses, HBV, EBV, HSV, HHV-8, Coxsackie viruses, polio viruses, cymegalo viruses influenze viruses, rubella viruses, papilloma viruses, dengue viruses, hanta viruses, arena viruses, bunya viruses, filo viruses), protozoa (plasmodia, toxoplasma, acanthamoeba), mykoplasma, fungi (candida, aspergillus), parasites (ascarida, echinococci, leishmania, loa-loa, onchocerca volvulus, masonella, brugia filaria, cysticercus, schistosoma, bancrofte-filaria) and infectious particles (prions). The antigenic determinants of infectious germs may also be of importance in the diagnosis and/or therapy of tumors as a target of the inventive IgY conjugates, for instance, in Burkift lymphoma, nasophariyngeal carcinoma, M. Hodgkin, T-cell lymphoma and immunoblastic lymphoma which are characterized by the expression of EBV-associated antigens (EBNA 1-3, LMP 1-2), in the Sarkosi-Sarkom and AIDS associated lymphoma, both of which are associated with HHV-8, or in adult T-cell leukemia (ATL) associated with HTLV-1 -infection, or in genital cancer associated with HPV.

d) Forms of Application:

Depending upon need, the inventive IgY conjugates may be applied in vitro or in vivo. In vivo, the application may take place externally or internally (e.g. in the context of photodynamic therapy as well as aparenterally (for instance in scintigraphy, SPECT, radio immunotherapy or immunotoxin therapy). As used herein, the term parenteral includes intravenous, intraarterial, subcutaneous, intracutaneous, intrathecal, interstitial, intracavernous and intralesional or intratumoral application procedures.

The invention will hereafter be explained and described in greater detail on the basis of detailed examples. The characteristics to be gleaned from the drawings and specification may in other embodiments of the invention be applied singly or in any desired combination with each other. The following applications and embodiments merely represent examples which in no manner exhaustively present the possibilities of the inventive IgY conjugates based upon SPF-chickens. A person skilled in the art will thus deduct further embodiments and applications which fall into the ambit of the claims of the present invention.

In the drawings:

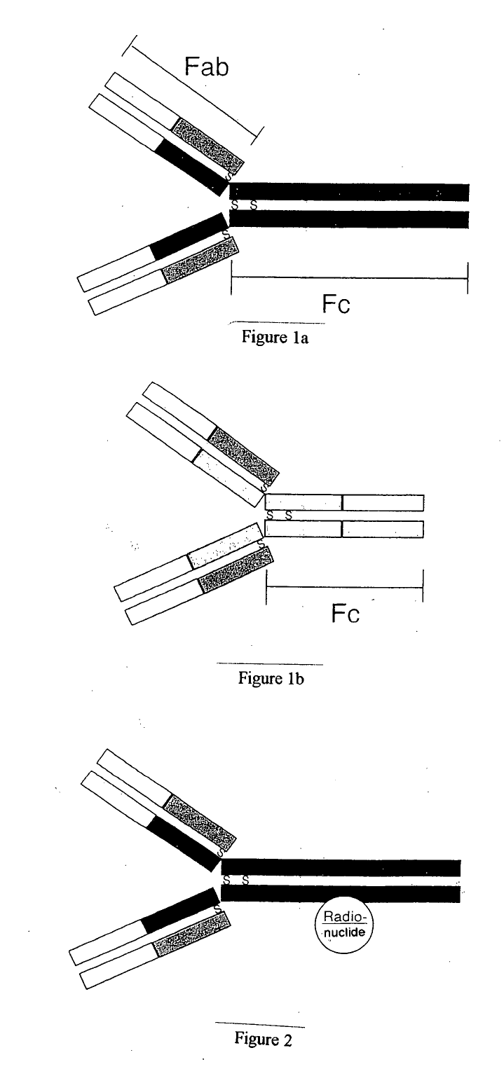

FIG. 1: depicts chicken immunoglobulin and, for comparison, mammalian immunoglobulin;

FIG. 1a: shows chicken IgY;

FIG. 1b: shows IgG of a mammal incl. Human;

FIG. 2: shows radio immunoconjugate based upon intact IgY;

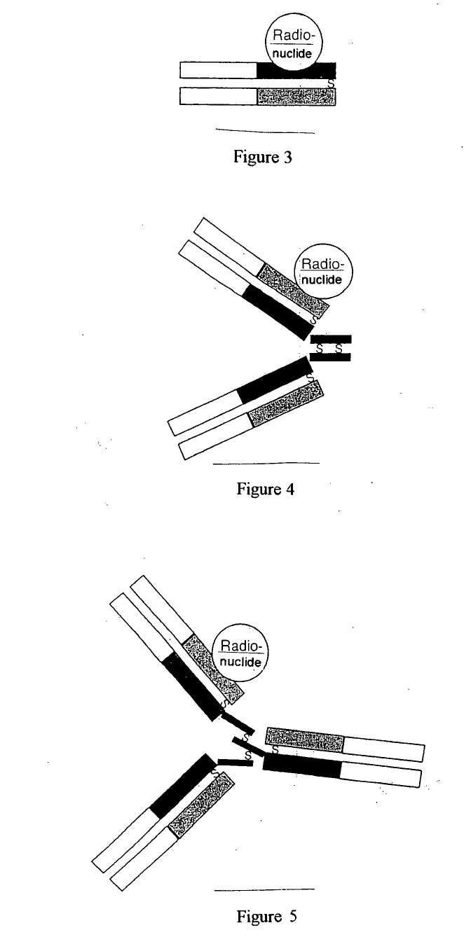

FIG. 3: shows radio immunoconjugate based upon a monovalent IgY Fab fragment;

FIG. 4: depicts radio immunoconjugate based upon a bivalent IgY-F(ab′)₂ fragment;

FIG. 5: depicts radio immunoconjugate based upon three fused IgY Fab fragments (trivalent Fab construct).

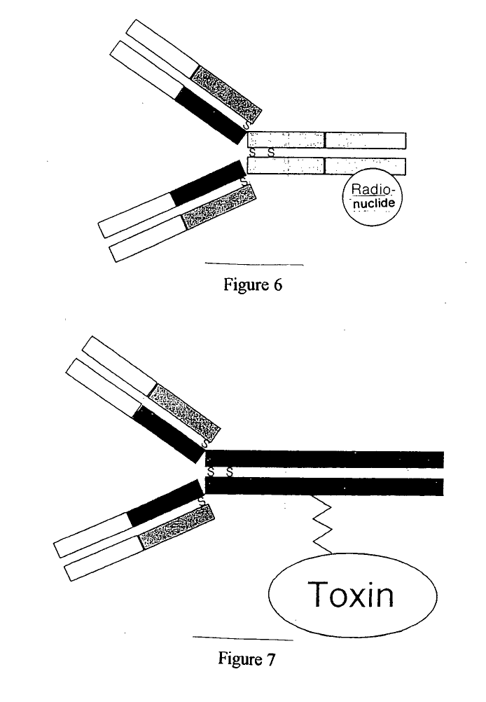

FIG. 6 depicts a radio immunoconjugate based upon humanized IgY (IgY-Fab+human Fc);

FIG. 7: depicts immunotoxin based upon intact IgY.

### EXAMPLE 1

For producing the inventive IgY conjugates SPF-chickens or transgenic SPF-chickens are immunized s.c or (preferably) i.m., preferably in their 14^{th} week of life, by 50-1,000 μg (preferably 50-200 μg) of the target antigen or a fragment thereof and complete Freund adjuvans. Thereafter, three boosters, until the maximum IgY titer has been reached, and three replenishments are carried out at 50-1,000 μg (preferably 50-200 μg) each over almost the entire laying period with the same antigen and incomplete Freund adjuvans, for maintaining the maximum IgY titer. The solution of each injection contains, for instance, 1 ml of antigen suspension and 0.5 ml of adjuvans. A preferred immunization schedule for chickens of SPF status is, therefore, of about the following chronological sequence:

Day 0*=1^{st} immunization (base immunization) i.m. injection;

Day 28:=2^{nd} immunization (1^{st}booster) i.m. injection;

Day 56:=3^{rd} immunization (2^{nd} booster) i.m. injection; IgY production;

Day 84:=4^{th} immunization (3^{rd} booster) i.m. injection; IgY production;

Day 168:=5^{th} immunization (1^{st} replen.) i.m. injection; IgY production;

Day 252:=6^{th} immunization (2^{nd} replen.) i.m. injection; IgY production;

Day 336:=7^{th}immunization (3^{rd} replen.) i.m. injection; IgY production;

Day 490:=End of use for IgY production.

*)=14^{th} week of life.

During a period of 12 to 14 months the eggs from chickens kept under SPF conditions may be collected and processed. The egg-yolk is initially separated from the egg-white and may be processed by standard procedures in accordance with Schade et al. (Chicken Egg Yolk Antibodies, Production and Application; Springer-Verlag, Berlin 2001 [1]). The decisive advantage of SPF-chickens is that these animals owing to their descent from SPF-chickens and being kept under SPF conditions, are not immunologically handicapped either because of immunizations or specific infectious diseases. Accordingly, these animals react with a particularly intensive immune response against the antigen with which they have been immunized so that the proportion of specific antibodies is higher by an order of magnitude (50-90% specific IgY) than by immunization of conventional chickens. Moreover, in these animals, following the boosters, the high titer of specific antibodies can be maintained stable by repeated replenishing immunization up to their 16^{th} week of life. This is of significance in respect of the IgY conjugates in accordance with the invention, for it is well-proven that by repeated stimulation of the immunological memory the lymphocyte populations change such that antibodies of particularly high affinity are formed. For that reason, the IgY compounds from eggs of SPF-chickens differ from IgY compounds derived from conventional chickens also in terms of their affinity to the specific antigen. On a molecular plane the higher affinity is conditioned by the structure of the hypervariable region of these antibodies, which matches the antibody better than does the structure of the hypervariable region of antibodies of lower affinity. The IgY conjugates in accordance with the invention derived from SPF-chickens in the final analysis reproducibly differ on a molecular plane (i.e. by the special structure of their hypervariable regions) from IgY compounds from conventional chickens.

### EXAMPLE 2

This example describes the production of humanized IgY from transgenic chickens. The transfection may take place in a manner known per se as described in U.S. Pat. Nos. 5,162,215, 6,020,465 and WO 00/75300. It may also be accomplished by microinjection into the sperms of a rooster. This characteristic is then inserted into the egg cell by artificial insemination and is passed on to the offspring. Alternatively, the transfection may be accomplished by retroviral infection of the sperms. A replication deficient retrovirus carries the human gene for the Fc fragment. The integration of the gene section of the constant human IgG domain into the immunoglobulin gene site of the SPF-chicken takes place by homologous recombination. The human “knock-in” gene targeting vector among others carries sequences which are homologous to those of the Exon of the constant human IgG regions and promoters. The deletion of the neomycin and of the thymidinkinase cassette and of the constant aviary IgY regions may be accomplished by transient Cre-recombinase expression. In this manner transgenic chickens result who pass on their genes for humanized IgY to their offspring by way of the germ path. The selection of the transgenic chickens May take place by proof of IgY with human constant regions in the blood or in the eggs of the chickens. ELISA, Western Blot or PCR are suitable for detecting the humanized antibodies.

### EXAMPLE 3

Intact antibodies, because of their slow elimination from the blood (HWZ: 1-3 days), in immunoscintigraphy often carry long examination times until an optimum contrast between the lesion to be presented and the background has been attained. In therapeutic applications the problem resides more likely in the fact that it is difficult to place intact antibodies into solid tumors or beyond the blood-brain barrier. For that reason conjugates on the basis of Fab fragments in certain situations offer decisive advantages. For producing the Fab conjugates SPF-chickens or transgenic chickens are immunized, as described in Example 1, by 50-1,000 μg (preferably 50-200 μg) of the target antigen or of a fragment thereof. The eggs are processed in the manner described supra and IgY is isolated at the specific antigen (target antigen) by affinity chromatography. The intact antibodies are then enzymatically split, for instance by pepsin in the area of the hinge region, so that Fab and Fc fragments are produced. The specific Fab fragments may be separated and isolated on the specific antigen from the Fc fragments and from non-specific Fab fragments, by affinity chromatography. The Fab fragments obtained in this manner are filtered in a sterile environment. They may be offered for clinical use in a labeling kit, which in a second piercing vial contains the reducing agent for the labeling, for conjugation with a short-lived diagnostic radionuclide (e.g. technetium 99m). Radio immunoconjugates may also be centrally labeled and distributed by their manufacturer with longer-life diagnostic and therapeutic radionuclides (e.g. iodine 131). In that case, transportation to a user takes place on the day of labeling in accordance with current regulations governing radiation protection, in order to minimize a loss of quality of the product as a result of decay of the radionuclide and dissociation of the antibody. Central labeling is also possible if the IgY compound is conjugated with a chemotherapeutic agent, toxin, chromium fluoride, fibrinolytic, enzyme or prodrug.

### EXAMPLE 4

Radiological sources of unclear dignity may nowadays be clarified in a differential diagnostic manner by granulocyte-scintigraphy. The murine monoclonal antibodies used at present recognize a non-specific-cross-reacting antigen expressed on promyelocytes, myelocytes and granulocytes, either NCA-95 (=CD66b) or NCA-90 (=CD66c). They are labeled with diagnostic radionuclides (gamma emitters) and applied intravenously. In the blood and at the source of infection they bond to granulocytes. In this manner infectious processes, e.g. osteomyelitis, can be shown scintigraphically. For producing the radio immunoconjugates in accordance with the invention, SPF-chickens are immunized and boostered with NCA-90 or NCA-95 or fragments thereof, in the manner described in Example 1. The NCA-90 or NCA-95-specific IgY is again isolated by affinity chromatography on the basis of NCA-90 and NCA-95, respectively. The sterile IgY may then be commercially distributed as a labeling kit, e.g. with 1 mg of isotonic NaCl and a second piercing vial with the reduction agent. For labeling the technetium 99 m. The anti-NCA-IgY-Tc-99 m prepared by the user in situ may then be intravenously applied after separation of unbound radionuclides by means of the column supplied with the kit and final quality control with respect to the radiochemical purity of the end product by high power liquid chromatography. (HPLC) or, preferably, thin-layer chromatography (DC). In contrast to conventional monoclonal antibodies which can recognize and bond with only one antigenic determinant, different antigenic determinants of NCA 90 or NCA 95 are simultaneously recognized by the polyclonal egg-yolk antibodies which result in an improved image rendition with a higher specific uptake relative to the background.

### EXAMPLE 5

IgY conjugates may also be used for detecting lymph nodes. Extensive lymph adenectomies as they are performed with malignant melanomata, mamma or prostate carcinomata are usually connected with a high morbidity. For that reason the detection of metastazation in the lymph nodes disposed in the side-stream area of a tumor constitutes highly important information for a therapeutic procedure. To date is it carried out with human colloidal albumen labeled with Tc 99m or with Tc 99m nanocolloid. The particle size is of 100-1,00 nm diameter. Following peritumoral injection of the radiocolloid three sequential planar images are prepared p. I. on the gamma camera within an interval of from 15 minutes to about 6 hours. The disadvantage of this method resides in the size of the particles and the lacking tumor specificity. On the one hand, a large part of the radioactivity remains at the site of the injection and complicates the presentation of the lymph nodes by excess radiation. On the other hand, lymph nodes are presented without their infection being proved thereby. If, by contrast, lymphocyte-specific or tumor-specific radio immunoconjugates are applied, those disadvantages are avoided in part or totally. The dissolved IgY or Fab conjugates drain more quickly through the lymph passages than colloidal albumin so that less residual activity remains at the site of the injection, and the lymph nodes are more intensely visible. Moreover, a specific detection results from the fact that the antibodies bond in the lymph nodes bond specifically to lymphocytes or metastasized tumor cells. These lymph nodes may also be detected intraoperatively and then resected with a suitable gamma probe. Alternatively or in addition to detecting the lymph nodes, these lymph nodes or lymph node metastases may be subjected to substantial selective radiation or chemotherapy if the diagnostically used IgY conjugate or a second IgY conjugate contains a therapeutic radionuclide (e.g. I 131) or toxin (e.g. Taxol®). The detection of lymph node metastases of a melanoma may be carried out by radio immunoconjugates against p210, a melanoma associated antigen (MAA). For producing the inventive radio immunoconjugates, SPF-chickens are immunized in accordance with Example 1 with 50-1,000 μg (preferably 50-200 μg) p210. IgY is isolated from the egg-yolk. The purified IgY may then be filtered in a sterile manner and lyophylized or suspended in sterile isotone PBS (pH 7.4). The commercially available compound kit contains, for instance, 100 mg specific IgY in a first piercing vial and, in a second piercing vial, the reducing agent for labeling with Tc 99m. Immediately prior to the planned examination the IgY compound is conjugated with 10-1,000 MBq (preferably 20-100 MBq) of Tc 99m. After peritumoral injection of the radio immunoconjugate planar images are taken p.i. in three planes for up to 24 hours (preferably three to six hours) under a gamma camera as well as, if required, a supplemental SPECT of the region in question. In addition, the affected lymph nodes may afterwards be intraoperatively detected with a suitable manual measuring device and selectively removed.

### EXAMPLE 6

The diagnostic of neuroendocrine tumors is carried out scintigraphically with radio immunoconjugates. Pheochromocytoma, neuroblastoma, carcinoids and paraganglioma develop from the cells of the neuroendocrine (APUD) system which are distributed throughout the entire organism. Thus, they generally possess, somatostatin receptors by means of which they may be scintigraphically rendered visible with the aid of octreotide (a somatostatin analog) labeled with indium 111. However, somatostatin analogs bond to the receptors at a 1:1 ratio. However, polyclonal IgY recognizes several antigenic determinants of the receptor. This leads to better targeting and diagnostics of improved image rendition. For producing the IgY compound against somatostatin receptors SPF-chickens are again immunized in accordance with Example 1. The specific IgY may be commercially offered as a labeling kit of the kind of Example 3, 4 or 5. It may then be labeled in situ by the user with a gamma emitter (e.g. technetium 99m). 1-24 hours following an i.v. application of 100 mg of specific IgY-Tc 99m (10-1,000 MBq, preferably 50-200 MBq) planar images are taken in two planes and, if necessary, a SPECT is performed. The neuroendocrine tumors which are usually hard to detect intraoperatively may be easily found during a following operative procedure and resected.

### EXAMPLE 7

A further application of the radio immunoconjugates is the treatment of non-Hodgkin-lymphomata. Non-Hodgkin lymphomata of low malignancy respond poorly ro chemotherapy. The radio immunotherapy has been shown to be particularly effective in such cases. In most studies, iodine 131 labeled murine monoclonal antibodies were used against CD20, an antigen which is expressed on the cell surface of 95% of all B-cell-lymphomata. However, monoclonal antibodies recognize but one antigenic determinant of this antigen. The bonding ratio is thus only 1:1. By contrast, improved targeting may be achieved by CD20-specific IgY which simultaneously recognizes several epitomes on the antigen. In this manner, the radiation exposure of the bone marrow is reduced, and higher radiation doses may be applied without necessitating a bone marrow transplant. A reduced radiation dose may also be achieved by pretargeting procedures with avidin or streptavidin labeled monoclonal antibodies and radioactively labeled biotin. By pretargeting, for which protection is also sought, the therapeutic effect of a radio immunotherapy with IgY may yet again be improved. To this end, SPF-chickens are immunized with 50-10,000 μg (preferably 50-200 mg) CD20 as in Example 1. During a laying period of 12 to 14 months, eggs are obtained from which the entire IgY is isolated and purified on CD20 by affinity chromatography. For pretargeting the specific IgY is filtered in a sterile manner, conjugated with avidin (or streptavidin) and lyophilized or dissolved, with further auxiliary agents, in isotonic NaCl (or PBS, pH 7.4). In this case, the labeling kit preferably contains a piercing vial with the IgY-avidin-conjugate (100 μg-5 mg, preferably 1 mg), a second piercing vial with biotin in a lyophilized or dissolved state (11-1,000 μg) and a third piercing vial with an oxidizing agent (e.g. lodogen) for the labeling with iodine 131 (1-4 GBq); furthermore, further auxiliary agents such as chromatographic column and DC kit. Alternatively, the biotin may be centrally labeled by the manufacturer and be delivered together with the IgY-avidin-conjugate. Treatment is initiated by intravenous application of the IgY-avidin-conjugates in order to label the lymphocytes. The intravenous application of the iodine 131-labeled biotin follows one or several days later. Alternatively, the radio immunotherapy may be carried out with an alpha emitter such as astatine 211.

### EXAMPLE 8

About half the mammary carcinoma possesses estrogen receptors and is dependent on hormones. Egg-yolk antibodies against estrogen receptors thus cannot only competitively inhibit the estrogen bonds at the receptors. As a radio immunoconjugate they are also suitable for detecting metastases and for internal radiation therapy. For immunizing the SPF-chickens, peptides with the extra-cellular antigenic determinants of the estrogen receptor are used. The immunization of the SPF-chickens as well as the preparation of the egg and purification of the IgY may be carried out in the manner of Example 1 and the production in accordance with one of the other Examples supra.

### EXAMPLE 9

Commercially available antibodies such as URO7 recognize kidney cell carcinoma and do not bond to healthy kidney tissue. Kidney cell carcinoma and their metastases may thus be specifically detected and/or treated by radio immunoconjugates. To identify a suitable antigen, the proteins of homogenized kidney carcinoma cell are electrophoretically opened and isolated. SPF-chickens are immunized with these antigens and the produced egg-yolk antibodies are examined with kidney carcinoma cells and physiological tissue with respect to specificity and cross reactivity. The selected antibodies, which recognize only malignant cells, are conjugated with a therapeutic radionuclide such as iodine 131 or with a cytotoxin such as ricin A and are used therapeutically.

### EXAMPLE 10

A further use of the radio immunoconjugates is seen in the treatment of HIV infections. In a HIV infection the retroviral genome is incorporated into the chromosomes of the host cell. A HIV infection cannot be cured with conventional replication inhibitors. The HIV replicating host cell expresses HIV structure protein (p24, gp120 and gp41) on its cell surface. Antibodies against these structure recognize these cells and bond therewith. If they are conjugated with a radionuclide or another cytotoxin, they can destroy these cells and, therefore, the production sites of HIV. A precondition for effective targeting of the cells is that the number of free cells in the blood is reduced, for instance by a complementary replication inhibition with antiretroviral medications. This opens up novel possibilities of effectively treating the HIV infection. For the reasons mentioned in the introduction, the use of polyclonal antibodies from specific IgY from SPF-chickens offers advantages over monoclonal antibodies of the kind described in DE 198 09 785. For producing the antibodies SPF-chickens are immunized with HIV p24, HIV-1 gp120 or HIV-1 gp41 as in Example 1. The eggs of the chickens kept under defined conditions are collected over a laying period of 12-14 months. The egg-yolk separated from the egg-white, and the IgY is isolated by one of the standard procedures. HIV-specific antibodies are purified by affinity chromatography on the specific antigen so that unspecific IgY is removed. The sterile IgY compound is conjugated with a therapeutic radionuclide (e.g. iodine 131 or astatine 211). Commonly, for each therapeutic application 100 μg of specific IgY is labeled with 1-4 GBq iodine 131. In case of an HIV infection, because of the relatively low number of infected cells (compared to the number of malignant degenerate cells of lymphoma) substantially lower activities may prove to be effective. The labeling may be carried out by different established procedures. IgY-iodine 131 could preferably be produced by the iodogen method. The yield after conjugation of the radio immunoconjugate as well as the purity of the radio immunoconjugate after separation of unbound radionuclides may be controlled by HPLC or DC. The radio immunoconjugate produced and/or sterile-filtered under sterile provisions may be infused as isotonic solution.

### EXAMPLE 11

In addition, IgY conjugates are to be used for the treatment of autoimmune diseases. CD-4 positive T-cells play an important role in the pathogenesis of autoimmune diseases and in rejection reaction in organ transplantations. With M. Crohn, monoclonal antibodies against the CD4 receptor displayed lupus erythematodes, rheumatoid arthritis, but insignificant effects after skin transplants in mice. In order to bring about an effective depletion of CD4 positive cells, the antibodies can be conjugated with a therapeutic radionuclide, a cytostatic agent or a toxin. SPF-chickens are immunized against CD4 or CD4 fragments according to Example 1; the entire IgY is isolated from the egg-yolk, and the CD4-specific IgY can be purified on CD4 by affinity chromatography. If necessary, the antibodies are cleaved enzymatically, and the aviary Fc fragments are replaced by human Fc fragments. The IgY compound may then be distributed for a (preferably intravenous) clinical application in an unconjugated state or labeled with a radionuclide as an Igy conjugate in accordance with the invention.

### EXAMPLE 12

The application of IgY conjugates for treating thromboses represent a further use. Thromboses are generally treated with systemically applied fibrinolytics (streptokinase, urokinase, rt-PA). Streptokinase is quickly broken by proteases of the body. Urokinase and rt-PA, however, are quickly metabolized by the liver. It is thus useful to fix fibrinolytics to the thrombus (Bode et al., J. Biol.Chem. 1989;264(2):944-8; Lijnen et al. Thromb Res 1990;57(3):333-42). SPF-chickens or transgenic chickens are immunized against human fibrin in the manner described in Example 1. The specific IgY is isolated and purified. If necessary, the antibodies are enzymatically cleaved and the Fc fragments are separated. Intact antibodies of Fab fragments (5-50 mg) are then conjugated with rt-PA (5-50 mg), preferably at a ratio of 1:1. These IgY conjugates may be offered in lyophilized state or in sterile solution for purposes of injection in suitably sized packages for treatment of deep crural thromboses, pulmonary embolisms, cerebro-vascular insults or myocardial infarcts. Alternatively, a second group of SPF-chickens may be immunized against rt-PA (preferably a region outside of the active center). The Fab fragments of the first group may then be hybridized with the Fab fragments of the second group. In this manner, fibrin-specific bifunctional Fab constructs may be produced to which rt-PA is conjugated by way of one branch.

### EXAMPLE 13

IgY compounds are conjugated with photosensitizers for photodynamic diagnostics or therapy. The photodynamic therapy represents a minimum invasive procedure chiefly for the treatment of carcinoma of the surface of the skin or mucous membrane, Kaposi sarcoma and melanoma. It is based upon a tumor-selective enrichment of light-sensitive substances which release their photodynamic action (fluorescence or formation of cell-damaging radicals and destruction of the tumor vascularization) by the absorption of light. The success of photodynamic therapy depends significantly upon the relative distribution of the photosensitizer in the malignant and healthy tissue. The fixation of the photosensitizer at the tumor cells may be significantly strengthened, for instance in cases of Esophagus carcinoma or malignant melanoma, by tumor-specific antibodies. Diagnosis or therapy may then take place with a suitable light source. In diagnostic applications violet light causes fluorescence of the malignant degenerate cells. In photodynamic therapy, malignant degenerate tissue becomes necrotic under the impact of red light.

1. IgY conjugates for producing a diagnostic or therapeutic agent for non-infectious inflammations, infectious diseases, clotting disturbances, autoimmune diseases and oncological diseases, consisting of a polyclonal IgY compound from eggs of specified pathogen-free (SPF) chickens in accordance with European Pharmakopeia and DAB10 or consisting of a polyclonal IgY compound from eggs of transgenic SPF-chickens, whereby the IgY conjugates contain polyclonal IgY with constant regions of human IgG and additionally consist of at least one signal and/or active agent and/or booster molecule.

2. The IgY conjugates of claim 1, whereby the polyclonal IgY compounds are purified egg-yolk antibodies (IgY), IgY fragments, Fab constructs or chimeric egg-yolk antibodies.

3. The IgY conjugates of claim 1, characterized by the fact that the IgY compounds used for the preparation contain humanized egg-yolk antibodies produced by chemical linking of variable regions of aviary antibodies with constant regions of human IgG.

4. The IgY conjugates of claim 1 and 2, characterized by the fact that the IgY compound consists of mono- or bi-valent antibody fragments, preferably Fab or F(ab)₂.

5. The IgY conjugates of claim 1 and 2, characterized by the fact that the IgY compound consists of bi-, tri- or polyspecific Fab constructs.

6. The IgY conjugates of claim 1, characterized by the fact that the signal agent is a diagnostic radionuclide, an enzyme, a stain and/or a photosensitizer.

7. The IgY conjugates of claim 1, characterized by the fact that the active agent is a therapeutic radionuclide, a cytostatic agent, a toxin, a chemotherapeutic agent, a prodrug, an enzyme, a photosensitizer and/or a fibrinolytic agent.

8. The IgY conjugates of claim 1, characterized by the fact that the IgY compound is conjugated with a booster molecule, preferably biotin or with a protein of the complement system.

9. The IgY conjugates of one or more of the preceding claims, characterized by the fact that the signal agent or active agent is bonded to the IgY compound by covalent bond or by an auxiliary agent such as a chelating agent, booster molecule and/or molecule of high affinity for the booster molecule, or by the variable region of one or more branches of a bi-, tri- or polyspecific IgY construct.

10. The IgY conjugates of one or more of the preceding claims, characterized by the fact the IgY compound is specific for tumor antigens, for hormones, for RNA- and/or DNA sections, for infectious germs or their (surface)-antigens, for leucocyte-antigens, for intracellular molecules, for receptor molecules or for clotting factors.

11. The IgY conjugates of one or more of the preceding claims, whereby in the sense of pretargeting the immunoconjugates are conjugated in vivo to the signal agent or active agent or booster molecule only after bonding to the specific antigen by way of secondary human anti-IgY antibodies.

12. The IgY conjugates of one or more of the preceding claims, whereby in the sense of pretargeting the immunoconjugates are conjugated in vivo to the signal agent or active agent or booster molecule antigen only after bonding to the specific antigen by way of secondary human anti-IgY antibodies.

13. The confectioning of IgY conjugates described in claims 1 to 12, whereby they are supplemented by at least a set for quality control by thin-layer-chromatography, separation columns, stands for the apparatus, washing and elution buffers.

14. The confectioning of claim 13, whereby the IgY conjugate is made available in lyophilized form or suspension in connection with additiives such as stabilizers set for an optimum pH value.

15. The confectioning of IgY conjugates of claim 13, whereby and application device is added.
